(19) United States
(12) Patent Application Publication
(10) Pub. No.: US 2017/0000799 A1
(43) Pub. Date: Jan. 5, 2017

(54) ORAL PHARMACEUTICAL COMPOSITION

(71) Applicant: LABORATORIOS DEL DR. ESTEVE, S.A., Barcelona (ES)

(72) Inventors: Ziauddin TYEBJI, Martorelles (ES); Meghal MISTRY, Ahmedabad (IN); Inder GULATI, New Delhi (IN); Vipan DHALL, New Delhi (IN)

(21) Appl. No.: 15/107,292

(22) PCT Filed: Dec. 19, 2014
(86) PCT No.: PCT/EP2014/078783

§ 371 Date: Jun. 22, 2016

(30) Foreign Application Priority Data
Dec. 23, 2013 (EP) 13382545.5

(51) Int. Cl. A61K 31/5377; A61K 9/00; A61K 45/06; A61K 31/4545; A61K 9/20

(52) U.S. Cl. CPC A61K 31/5377; A61K 31/4545; A61K 9/2054; A61K 9/2013; A61K 9/2018; A61K 9/2095; A61K 45/06; A61K 9/0053; A61K 9/20

(57) ABSTRACT

The present invention relates to solid particles of a poorly soluble drug, pharmaceutical compositions comprising them and processes for their preparation. The compositions according to the present invention show an improved dissolution profile while being stable.

### FIELD OF INVENTION

The present invention relates to solid particles of a poorly soluble drug, pharmaceutical compositions comprising them and processes of preparing such compositions.

### BACKGROUND OF THE INVENTION

Although pharmaceuticals may be administered in a variety of ways, ease of administration means that oral drug delivery is the preferred administration route. Solid oral dosage forms are particularly preferred since these offer greater drug stability, more accurate dosing, and ease of administration. However, for the treatment to be effective the oral dosage form must readily release the drug for its absorption.

A great number of new pharmaceutical drug substances are poorly water soluble and are therefore not well-absorbed after oral administration. Moreover, absorption of most drugs takes place in the upper small intestine and is greatly reduced after the ileum, meaning that the absorption window is small. One of the current challenges in the pharmaceutical industry is the development of strategies that improve drug bioavailability, for example through the development of fast release formulation which ensure that the drug is released in the short timeframe required for its uptake, or by improving drug solubility.

Various techniques are employed to increase the solubility of the drug which include, but are not limited to, decreasing the particle size, complexation, formation of a solid solution, changing the surface characteristics of the particles and incorporation of drug particles into colloidal systems like nanoparticles and liposomes.

5-Cloro-N-[2-oxo-3-[4-(3-oxomorpholin-4-yl)phenyl]-1,3-oxazolidin-5(S)-ylmethyl]-thiophene-2-carboxamide is a low molecular, orally administrable inhibitor of the blood coagulation factor Xa, investigated for the prophylaxis and/or treatment of various thromboembolic diseases (see WO 01/47919) and known under the INN rivaroxaban or under the trade name Xarelto®. Rivaroxaban, as well as some other direct factor Xa inhibitors (dabigatran, apixaban, ximelagatran, otamixaban, edoxaban, betrixaban), is practically insoluble in water (<100 mg/l at 25° C.), and moreover, has a low solubility in many organic solvents, including ethanol, and hence presents significant challenges to formulators. Further, since rivaroxaban is a low dose drug, there are further challenges as to achieving uniform distribution of the drug in a tablet.

In addition, different solid-state forms of the same chemical compound may have different chemical and physical properties that can have an impact upon drug product bioavailability and stability.

In the case of rivaroxaban, the holder of the product (see EMA website) stated that polymorphism has been tested and polymorph I is the thermodynamically stable and the one that has been used in all tablet formulations.

The prior art discloses various approaches for formulating rivaroxaban to improve its bioavailability. WO 2005/060940 teaches the use of the wet granulation technique in combination with the use of hydrophilic matrix formers in order to hydrophilize the rivaroxaban and to improve bioavailability.

US 2010/0151011 discloses solid pharmaceutical dosage forms of rivaroxaban in multiparticulate form, which can be prepared by melting the active agent with one or more excipients. The process yields a melt or melt extrudate which, following milling, forms granules or powders that can be encapsulated, or further processed with other excipients to form granulates that can be compressed into tablets. However, melt processing is not a particularly desirable procedure as it restricts the excipients that can be used and further entails operation at suitably high temperatures to enable the production of a melt. This increases the risk of drug decomposition and polymorphic changes, as well as drug-excipient reactions, potentially leading to the presence of decomposition products in the final dosage form. US 2010/0151011 also discloses a method whereby rivaroxaban is dissolved together with an excipient (polyvinylpyrrolidone) in glacial acetic acid at high temperature, distilled, and dried. The resulting granules are ground and sieved. As discussed above, this method suffers from fact that there is a lack of suitable solvents that can be used to dissolve rivaroxaban. Acetic acid is a high boiling solvent that needs to be removed by evaporation. Hence, this process is highly energy intensive, and is not suitable for large scale manufacture.

WO 2010/003641 discloses pharmaceutical compositions of rivaroxaban comprising a solubilizer and a pseudo-emulsifier as excipients. The solubilizer can be a surfactant, and the pseudo-emulsifier is a natural product, such as a natural gum. The compositions can be prepared by dry granulation, by pellet layering to form a multiparticulate, by melting followed by grinding, or by co-precipitation with an antisolvent. These processes are said to form primary pharmaceutical compositions in the form of granules which are then further processed into a dosage form by mixing with further excipients and compressing to provide tablets. According to the disclosure of this publication, the compositions are preferably immediate release formulations. The processes disclosed in this publication involve the production of an intermediate product, namely granules before these are compressed to form a tablet, and hence involve multiple steps. Moreover, processes such a co-precipitation use large volumes of solvent, which is not economical, nor desirable, from an environmental perspective.

WO 2010/146179 discloses solid pharmaceutical compositions of rivaroxaban, prepared by dry mixing or dry granulation of the rivaroxaban with at least one excipient, co-milling rivaroxaban with the excipients, hot melt granulation with a molten excipient, or hot melt extrusion with an excipient. The mixture may then be agglomerated, granulated with a granulation liquid, or milled before compressing to form a tablet. As discussed above, melt processing is not a desirable process for large scale manufacture in view of the energy requirements and the potential for prolonged heating to cause degradation of the active agent. Further, co-milling is a very energy intensive process. Moreover optimum blend uniformity can be difficult to achieve using co-milling and dry granulation processes.

The methods described in the prior art involve undesired steps that raise significant disadvantages to the overall tablet preparation process. It would therefore be desirable to provide compositions of drugs that have low water solubility, or drugs that are practically insoluble in water wherein the compositions have good blend uniformity, and which can achieve consistent release and dissolution profiles and moreover have a good bioavailability of the drug. It would also be desirable to provide a composition that can be easily manufactured by a simple process, wherein the risk of product degradation is reduced. Preferably the process avoids the use of process steps that are susceptible to causing polymorphic changes or degradation of the active agent (e.g. melt processing and co-precipitation). It would be further desirable to provide a process which can easily be adapted to provide immediate- or modified-release of the active agent. It would be a further desirable if the use of organic solvents and high temperatures are minimized, thus providing environmental and economical advantages. The present invention aims to achieve at least one or more of these objectives.

### SUMMARY OF THE INVENTION

The inventor of the present invention has surprisingly found that a solid particle of a poorly soluble drug, having an average particle size of 100 μm or less, wherein a solubilizer is adsorbed on the surface of the poorly soluble drug, allows the improvement in the solubility of the drug without affecting the drug stability and the drug polymorphism. The pharmaceutical formulation which comprises the said solid particle shows an immediate release of the active ingredient and ensures an effective amount of the drug released in less than 1 hour after intake.

In particular, one aspect of the present invention is directed to a solid particle of a poorly soluble drug, having an average particle size of 100 μm or less, wherein a solubilizer is adsorbed on the surface of the poorly soluble drug.

In an embodiment, the poorly soluble drug is selected from an anticoagulant agent selected from Xa inhibitors such as rivaroxaban, dabigatran, apixaban, ximelagatran, otamixaban, edoxaban, betrixaban, preferably, the Xa inhibitor is rivaroxaban or apixaban. In a further embodiment, the poorly soluble drug is in micronized form, preferably having an average particle size of less than 100 μm, preferably less than 50 μm, preferably less than 30 μm, preferably less than 20 μm and more preferably less than 10 μm.

In a second aspect, the invention relates to a process to prepare the said solid particle.

In a further aspect, the invention relates to an oral pharmaceutical composition comprising the aforementioned solid particles with at least one pharmaceutically acceptable excipient, preferably the solid particles of the poorly soluble drug comprise an anticoagulant agent, and more preferably the solid particles comprise rivaroxaban or apixaban.

In an embodiment, the pharmaceutical composition is a tablet, a minitablet or an orodispersible tablet.

In a further aspect, the invention relates to a process for producing the said oral pharmaceutical composition.

Finally, the invention in one of its aspects, relates to the oral pharmaceutical composition, wherein the solid particles of the poorly soluble drug comprise an anticoagulant agent, for use in the prophylaxis and/or treatment of thromboembolic diseases.

This aspects and preferred embodiments thereof, are additionally also defined in the detailed description as well as in the claims.

### BRIEF DESCRIPTION OF THE DRAWINGS

FIG. 1 Shows the dissolution profile of tablets prepared according to the invention compared with the dissolution profile of tablets prepared by direct compression.

### DETAILED DESCRIPTION OF THE INVENTION

The technical problem underlying the present invention is to provide an alternative solution to solubility improvement of poorly soluble drugs in order them to be used in pharmaceutical compositions warrantying its dissolution profiled while not affecting its stability.

The compositions of the present invention are stable, easy to prepare, and provide the desired in-vitro release of the active ingredient in spite of its low solubility. One additional advantage of the formulation of the invention is that it ensures the polymorphic stability of the active ingredient.

One aspect of the present invention is directed to a solid particle of a poorly soluble drug, having an average particle size of 100 μm or less, wherein a solubilizer is adsorbed on the surface of the poorly soluble drug.

According to the BCS classification system, the term “poorly soluble drug” is understood as a drug not being soluble in 250 ml of aqueous media over the range pH 1-pH 7.5. The drug can be selected from a variety of known drugs including:

anti-infectious drugs such as acyclovir, darunavir, indinavir, tenofovir, efavirenz, fluconazole, itraconazole, nelfinavir, nevirapine, praziquantel, ritonarvir.

antineoplasic drugs such as bicalutamide, cyproterone, gefitinib, imatinib and tamoxifen.

cardiovascular agents such as acetazolamide, atorvastatin, benidipine, candesartan, carvedilol, clopidogrel, ezetimibe, irbesartan, nifedipine, nilvadipine, nisoldipine, simvastatin, telmisartan, ticlopidine, valsartan, verapamil, warfarin.

antithrombotic agents such as rivaroxaban, apixaban,

Preferably, the poorly soluble drug is anticoagulant agents selected from Xa inhibitors such as rivaroxaban, apixaban, dabigatran, ximelagatran, otamixaban, edoxaban and betrixaban. Rivaroxaban or apixaban is a preferred drug.

In this regard it is noted that rivaroxaban or its solvates or hydrates as well as pharmaceutical acceptable salts thereof, used in the present invention is preferably obtained according to the procedures as outlined in WO 01/47919. The solid form thus obtained has been described in WO 2007/037132 as crystalline form I. Rivaroxaban as used in the present invention can be micronized or non-micronized. Rivaroxaban is preferably provided in a micronized form, preferably having an average particle size of less than 100 μm, preferably less than 50 μm, preferably less than 30 μm, preferably less than 20 μm and more preferably less than 10 μm.

The term “average particle size” as used herein has its conventional meaning as known to the person skilled in the art and can be measured by art-known particle size measuring techniques such as, for example, sedimentation files flow fractionation, photon correlation spectroscopy, laser diffraction or disk centrifugation. The average particle sizes mentioned herein relates to weight distributions of the particles. In that instance, by “average particles size of less than 100 μm” it is meant that at least 90% of the weight of the particles have a particle size below 100 μm, and the same applies to the other particle sizes mentioned.

The term “particle” as used herein is intended to mean any solid or semi-solid portion of a substance or a composition having defined physical boundaries. In particular, the present invention uses “particle” with the meaning of powder. The solid particles of the invention contain a poorly soluble drug adsorbed with a solubilizer. The solid particles of the invention are free of other pharmaceutical excipients different than solubilizers. These solid particles have an average particle size of less than 100 μm, preferably less than 50 μm, preferably less than 30 μm, preferably less than 20 μm and more preferably less than 10 μm.

The ratio of the poorly soluble drug contained in the fine particles of the present invention in terms of the total of solid particles should be 0.1 to 99.9 wt %, preferably 0.5 to 99 wt %, particularly 10 to 95% wt.

Unless otherwise stated, all amounts are expressed herein as percentage by weight in a dry matter basis.

The term “solubilizer” as used herein is intended to mean substances used to improve solubility. Examples of solubilizers include, but are not limited to, polyethylene oxide, hydroxyalkyl cellulose, hydroxypropylalkyl cellulose, polyvinyl alcohol, polyvinylpyrrolidone, copovidone, sodium carboxymethyl cellulose, carbopol, sodium alginate, xanthan gum, locust bean gum, cellulose gum, gellan gum, tragacanth gum, karaya gum, guar gum, acacia gum, poloxamer, cyclodextrin, dextrin derivatives, surfactants and mixtures thereof and other materials known to those ordinary skill in the art.

The term “surfactant” as used herein is intended to mean substances used to reduce the surface tension of the aqueous solutions comprising them. Surfactants are classified as anionic, cationic and nonionic. Examples of surfactants include, but are not limited to, self-emulsifying glyceryl monooleate, docusate sodium, emulsifying wax BP, sodium lauryl sulfate (SLS), benzethonium chloride, cetrimide, cetylpyridinium chloride, lauric acid, myristyl alcohol, sorbic acid, emulsifying wax, glyceryl monooleate, phospholipids, polyoxyethylene alkyl ethers (macrogol cetostearyl ether, macrogol lauryl ether, macrogol oleyl ether, macrogol stearyl ether), polyoxyethylene castor oil derivatives (macrogolglycerol ricinoleate, macrogolglycerol hydroxystearate), polyoxyethylene sorbitan fatty acid esters (polysorbate 20, 40, 60, and 80), polyoxytehylene stearates, polyoxylglycerides (caprylocaproyl polyoxylglycerides, lauroyl polyoxylglycerides, linoleoyl polyoxylglycerides, oleoyl polyoxylglycerides and stearoyl polyoxylglycerides), sorbitan esters (sorbitan laurate, sorbitan oleate, sorbitan palmitate, sorbitan sesquioleate, sorbitan stearate, sorbitan trioleate), triethyl citrate and mixtures thereof and other surfactants known to those skill in the art. Preferably, the surfactant is selected from sodium lauryl sulfate, polyoxyethylene sorbitan fatty acid esters and polyoxylglycerides.

In the solid particle of the present invention, the solubilizer is adsorbed on the surface of the poorly soluble drug. Surprisingly, the absorption significantly improves wettability of the drug in the aqueous media while in turns improves solubility at gastrointestinal tract pH.

One exemplary method for forming adsorbates of the present invention is solvent processing. Solvent processing consists of dissolution of the solubilizer in a solvent and pouring/spraying it onto the drug followed by removal of the solvent by evaporation or by mixing with a non-solvent. Preferably, the removal of the solvent results in a solid adsorbate. The resulting adsorbates of the present invention have a great physical stability and dissolution performance.

The adsorption of the solubilizer can be carried out in a polar or a non-polar solvent, protic or aprotic. Suitable solvents include for instance, alcohols, acetone, acetonitrile, water or mixtures thereof. For environmental reasons, the preferred solvent is water.

An aspect of the invention is directed to a process for producing the said solid particles characterized in that it comprises the following steps:
a. the solubilizer is dissolved or suspended in a polar or a non-polar solvent, protic or aprotic or mixtures thereof
b. the solution or suspension obtained in step (a) is poured or sprayed on to the surface of the poorly soluble drug.

In a preferred embodiment, the solvent is water.

The adsorption of the solubilizer on the poorly soluble drug is carried out by pouring an aqueous solution of the solubilizer on to the surface of the poorly soluble drug (step b) and drying at a temperature ranging from 35 to 65° C. Then, the solid particles are sieved in order to obtain a fine powder.

In another aspect, the invention is directed to an oral pharmaceutical composition comprising the said solid particles with at least one pharmaceutical excipient. The pharmaceutically acceptable excipients that may be incorporated in the composition of the present invention include, but are not limited to, fillers, binders, disintegrants, lubricants, and the like or combinations thereof.

Examples of fillers include, but are not limited to, sucrose, glucose, lactose, mannitol, xylitol, dextrose, microcrystalline cellulose, coprocessed microcrystalline cellulose, maltose, sorbitol, calcium phosphate, calcium sulfate, carraggenan, chitosan, pectinic acid, sodium alginate, magnesium aluminium silicate and the like and also, mixtures thereof. Preferably the fillers are lactose and microcrystalline cellulose.

The percentage of the filler in the formulation according to this invention is from about 20% to about 80%, preferably about 30% to about 70%, more preferably about 40 to about 60% by weight with respect to the total weight of the formulation.

Examples of binders include, but are not limited to, celluloses such as microcrystalline cellulose, modified celluloses (such as low substituted hydroxypropyl cellulose, hydroxypropyl cellulose (or HPC), hydroxypropyl methylcellulose (or HPMC or hypromellose), hydroxyethylcellulose, hydroxyethyl methylcellulose, ethyl cellulose, cellulose gum, xanthan gum, sugars (such as sucrose, glucose, amilose, maltodextrin, dextrose and the like), starches such as corn or potato starch partially pregelatinized starches (such as Starch 1500), polyvinyl acetate (Kollicoat SR), polyvinyl alcohol-polyethylene glycol graft copolymer (Kollicoat IR), copovidone, cross-linked polyvinylpyrrolidone, acrylic acid polymer (Carbopol), poloxamer, polycarbophil, polyethylene oxide, polyethylene glycol or a combination thereof. Preferably, the binder is hydroxypropyl methylcellulose.

The preferred percentage of binder in the formulation according to this invention is from about 0.1% to about 30%, preferably about 0.1% to 10%, more preferably about 0.1% to 5% by weight with respect to the total weight of the dry matter of the formulation.

The following are examples of useful disintegrants: starches such as corn or potato starch, modified starches (such as sodium starch glycolate) and partially pregelatinized starches (such as Starch 1500); polyvinylpyrrolidones, including modified polyvinylpyrrolidones (such as crospovidone, polymerized under conditions that promote crosslinking), crosslinked carboxymethylcellulose sodium (crosscarmellose sodium), ion exchange resins (such as Polacrilin potassium, Polacrilex) Neusilins, low substituted hydroxypropyl cellulose or a combination thereof.

The preferred percentage of disintegrant in the formulation according to this invention is from about 0.1% to about 20%, preferably about 1% and 18%, more preferably about 5 to 15% by weight with respect to the total weight of the dry matter of the formulation.

Examples of lubricants include, but are not limited to, calcium stearate, glyceryl monostearate, glyceryl palmitostearate, magnesium stearate, sodium stearyl fumarate, talc powder, colloidal silicon dioxide, stearic acid or a combination thereof.

The preferred percentage of lubricant in the formulation according to this invention is from about 0.5% to about 10% by weight with respect to the total weight of dry matter of the formulation. The most preferred percentage is about 1.0% to 7.0% by weight with respect to the total weight of dry matter of the formulation.

In addition, the formulation of the present invention may further comprise a coating layer to provide color, stability, release control or taste masking of a drug.

Examples of coating agent that may be used in such coating process include, but are not limited to, cellulose derivatives, vinyl derivatives, polymers and copolymers, gums, acrylic or methacrylic acid polymers, copolymers, esters or derivatives thereof, and the like or combinations thereof. Cellulose derivatives that may be employed, include, but are not limited to, methylcellulose, hydroxypropylmethylcellulose, hydroxypropyl cellulose, hydroxyethyl cellulose, hydroxymethyl cellulose, ethylcellulose, hydroxypropyl ethylcellulose, carboxymethylethyl cellulose, carboxy ethylcellulose, carboxymethyl hydroxyethylcellulose, hydroxyethylmethyl carboxymethyl cellulose, hydroxyethyl methyl cellulose, carboxymethyl cellulose, methylhydroxyethyl cellulose, methylhydroxypropyl cellulose, carboxymethyl sulfoethyl cellulose, sodium carboxymethyl cellulose, and the like or combinations thereof. Vinyl derivatives, polymers and copolymers thereof that may be employed include, but are not limited to copolymers of vinyl pyrrolidone, copolymers of polyvinyl alcohol (Kollicoat IR), polyvinylpyrrolidone or combinations thereof. Gums that may be employed include, but are not limited to, gum arabic, alginates, guar gum, locust bean gum, carrageenan, pectin, xanthan gum, gellan gum, maltodextrin, galactomannan, karaya, and the like, or combinations. Acrylic or methacrylic acid polymers, copolymers, esters or derivatives thereof, that may be employed include, but are not limited to, a) copolymer formed from monomers selected from methacrylic acid, methacrylic acid esters, acrylic acid and acrylic acid esters b) copolymer formed from monomers selected from butyl methacrylate, (2-dimethylaminoethyl)methacrylate and methyl methacrylate c) copolymer formed from monomers selected from ethyl acrylate, methyl methacrylate and trimethylammonioethyl methacrylate chloride or d) copolymers of acrylate and methacrylates with/without quarternary ammonium group in combination with sodium carboxymethylcellulose, e.g. those available from Rohm GmbH under the trademark Eudragit® like Eudragit EPO (dimethylaminoethyl methacrylate copolymer; basic butylated methacrylate copolymer), Eudragit RL and RS (trimethylammonioethyl methacrylate copolymer), Eudragit NE30D and Eudragit NE40D (ethylacrylate methymethacrylate copolymer), Eudragit RD 100 (ammoniomethacrylate copolymer with sodium carboxymethylcellulose); or the like or any combinations thereof.

The non-polymeric pharmaceutically acceptable agents used for the coating layer include, but are not limited to fatty acids, long chain alcohols, fats, in particular mono-, di- or triesters of glycerol and fatty acids, waxes, and the like, or combinations thereof. Fatty acids that may be employed include, but are not limited to, decenoic acid, docosanoic acid, stearic acid, palmitic acid, lauric acid, myristic acid, hydrogenated palm kernel oil, hydrogenated peanut oil, hydrogenated palm oil, hydrogenated rapeseed oil, hydrogenated rice bran oil, hydrogenated soybean oil, hydrogenated sunflower oil, hydrogenated castor oil, hydrogenated cottonseed oil, and the like, and mixtures thereof. Long chain monohydric alcohols that may be employed include, but are not limited to, cetyl alcohol, stearyl alcohol and mixtures thereof. Waxes that may be employed include, but are not limited to, spermaceti wax, carnauba wax, Japan wax, bayberry wax, flax wax, beeswax, Chinese wax, shellac wax, lanolin wax, sugarcane wax, candelilla wax, paraffin wax, microcrystalline wax, petrolatum wax, carbowax, glyceryl monostearate, glyceryl distearate, glyceryl tristearate, glyceryl dipalmitate, glyceryl tripalmitate, glyceryl monopalmitate, glyceryl dilaurate, glyceryl trilaurate, glyceryl monolaurate, glyceryl trimyristate, glyceryl monodecenoate, glyceryl didecenoate, glyceryl tridecenoate, glyceryl behenate and the like, or mixtures thereof.

In a further embodiment, in addition to polymeric or non-polymeric pharmaceutically acceptable agent or any combination thereof, the coating layer may optionally further comprise one or more pharmaceutically acceptable excipients such as, but not limited to, plasticizer, anti-tacking agent, pigment, and the like, or combinations thereof. A plasticizer that may be employed includes, but is not limited to, triethyl citrate, acetyl triethyl citrate, propylene glycol, polyethylene glycol, acetyl tributyl citrate, acetylated monoglycerides, glycerin, triacetin, phthalate esters (e.g., diethyl phthalate, dibutyl phthalate), castor oil, sorbitol and dibutyl seccate or a combination thereof. An anti-tacking agent that may be employed includes, but is not limited to, talc, or glyceryl monostearate. A pigment such as, but not limited to, titanium dioxide, iron oxide, or a mixture thereof may be employed.

The term “composition” or “formulation” has been employed interchangeably for the purpose of the present invention.

In one embodiment, the composition of the present invention can be in the form of capsules, tablets, minitablets, stick formulation, orodispersible tablets, dry suspension for reconstitution, powder or granule for solution or suspension, granules, and the like or any combinations thereof. In a preferred embodiment of the invention, the dosage form is a tablet, a minitablet or an orodispersible tablet. Depending of the final dosage form the compositions of the present invention may comprise appropriate pharmaceutically acceptable excipients such as those mentioned above or some additional ones such as, but not limited to, sweeteners, flavors, colorants and the like or combinations thereof. Further it is contemplated within the scope of the invention that the dosage form can be encapsulated or coated. In one preferred embodiment, the composition of the present invention is in the form of a tablet. In a further embodiment, the compositions of the present invention may be manufactured using conventional techniques known in the art.

In another aspect, the present invention provides a process for the preparation of a composition comprising the solid particles of the solubilizer adsorbed on to the poorly soluble drug with at least one pharmaceutically acceptable excipient. In a particular embodiment of the present invention, the said composition is a tablet prepared by direct compression.

The process for producing the oral pharmaceutical composition of the invention comprises the following steps:

a. preparing the said solid particles

b. mixing the particles of step a. with at least one pharmaceutical excipient

In the particular case that the dosage form is a tablet, the process further comprises pressing the mixture obtained in step b. in to a tablet.

In particular, the process of preparing the composition of the invention comprises the following steps:
(i) preparing a solution of the solubilizer in a solvent
(ii) pouring or spraying the solution of step (i) onto the surface of the poorly soluble drug
(iii) drying and sifting the mixture obtained in step (ii)
(iv) sieving to obtain a fine powder
(v) blending the fine powder of step (iv) with the pharmaceutical excipients
(vi) lubricating the blend of step (v)
(vii) tableting
(ix) optionally, film coating

In a further aspect, the present invention provides the pharmaceutical composition of the present invention, wherein the solid particles of the poorly soluble drug comprise an anticoagulant agent, for use in the manufacture of a medicament for the prophylaxis and/or treatment of thromboembolic diseases.

The invention is further illustrated by the following examples, which are for illustrative purposes and should not be construed as limiting the scope of the invention in any way.

### EXAMPLES

### Comparative Example 1 (Direct Compression-No Adsorbed Particles)

| Ingredients | mg/tablet | % w/w |
|---|---|---|
| Rivaroxaban | 10 | 11.76 |
| Microcrystalline Cellulose | 40 | 47.06 |
| Lactose Monohydrate | 26.5 | 31.18 |
| Croscarmellose Sodium | 3 | 3.53 |
| Hydroxypropylmethylcellulose | 3 | 3.53 |
| Sodium lauryl sulfate | 2 | 2.35 |
| Magnesium stearate | 0.5 | 0.59 |
| Purified Water | q.s. | q.s. |
| Total | 85 | 100.00 |

Procedure:

Brief description of process is as under:
1. Mixing of pre sieved Rivaroxaban, Sodium lauryl sulphate, Lactose Monohydrate, Hydroxypropylmethylcellulose.
2. Mixing the mixture of Step 1 with microcrystalline cellulose and croscarmellose sodium
3. Lubricate the mix of step 2 with magnesium stearate.
4. Compress the blend into tablets

### Example 2

| Ingredients | mg/tablet | % w/w |
|---|---|---|
| Rivaroxaban | 10.04 | 11.76 |
| Microcrystalline Cellulose | 40 | 47.06 |
| Lactose Monohydrate | 26.5 | 31.18 |
| Croscarmellose Sodium | 3 | 3.53 |
| Hydroxypropylmethylcellulose | 3 | 3.53 |
| Sodium lauryl sulfate | 2 | 2.35 |
| Magnesium stearate | 0.5 | 0.59 |
| Purified Water | q.s. | q.s. |
| Total | 85 | 100.00 |

Procedure:

Brief description of the process is as under:
1. Dissolve SLS in water.
2. Spray/pour solution of step 1 on to Rivaroxaban
3. Dry the mixture and sieve to break the lumps/agglomerates.
4. Mix the powder of step 3 with lactose, microcrystalline cellulose, cross carmellose sodium.
5. Lubricate the mix of step 4 with magnesium stearate.
6. Compress the blend into tablets

### Example 3

| Ingredients | mg/tablet | % w/w |
|---|---|---|
| Rivaroxaban | 10.04 | 11.8 |
| Microcrystalline Cellulose | 39.99 | 47.00 |
| Lactose Monohydrate | 26.5 | 31.2 |
| Croscarmellose Sodium | 3 | 3.5 |
| Hydroxypropylmethylcellulose | 3 | 3.5 |
| Polysorbate 80 | 2 | 2.4 |
| Magnesium stearate | 0.5 | 0.6 |
| Purified Water | q.s. | q.s. |
| Total | 85 | 100.00 |

Procedure:

Brief description of the process required
1. Dissolve Polysorbate 80 in water.
2. Spray/pour solution of step 1 on to Rivaroxaban
3. Dry the mixture and sieve to break the lumps/agglomerates.
4. Mix the powder of step 3 with lactose, microcrystalline cellulose, cross carmellose sodium.
5. Lubricate the mix of step 4 with magnesium stearate.
6. Compress the blend into tablets

### Example 4

### Comparison Between Dissolution Profiles of Example 1 and 2 Formulations

The dissolution of Example 1 and 2 tablets is performed in 900 ml, pH 4.5 acetate buffer containing 0.4% sodium lauryl sulphate, in USP-II apparatus at 75 RPM.

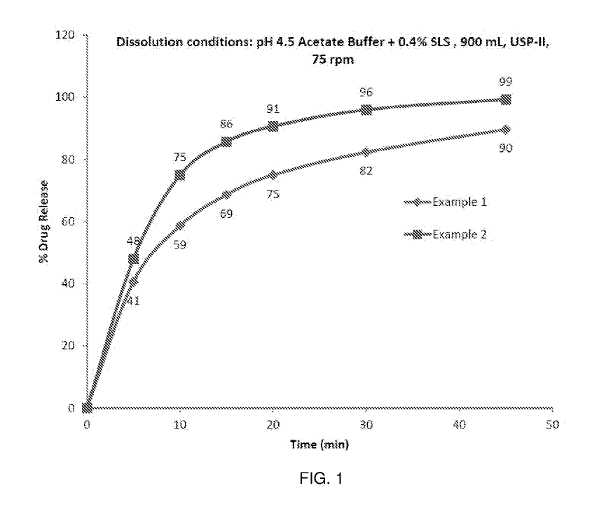

The dissolution profile clearly demonstrates that tablets prepared according to the present invention improve the solubility of the drug (FIG. 1).

### Example 5

| Ingredients | mg/tablet | % w/w |
|---|---|---|
| Apixaban | 2.5 | 2.5 |
| Microcrystalline Cellulose | 41 | 41 |
| Lactose Monohydrate | 51.5 | 51.5 |
| Croscarmellose Sodium | 4 | 4 |
| Sodium lauryl sulfate | 0.5 | 0.5 |
| Magnesium stearate | 0.5 | 0.5 |
| Purified Water | q.s. | q.s. |
| Total | 100 | 100 |

Procedure:

Brief description of the process required
1. Dissolve Sodium Lauryl Sulfate in water.
2. Spray/pour solution of step 1 on to Apixaban
3. Dry the mixture and sieve to break the lumps/agglomerates.
4. Mix the powder of step 3 with lactose, microcrystalline cellulose, cross carmellose sodium.
5. Lubricate the mix of step 4 with magnesium stearate.
6. Compress the blend into tablets

1.-16 (canceled)

17. A solid particle of a poorly soluble drug, having an average particle size of 100 μm or less, wherein a solubilizer is adsorbed on the surface of the poorly soluble drug.

18. The solid particle according to claim 17, wherein the poorly soluble drug is selected from an anticoagulant agent selected from factor Xa inhibitors.

19. The solid particle according to claim 18, wherein the poorly soluble drug is rivaroxaban.

20. The solid particle according to claim 18, wherein the poorly soluble drug is apixaban.

21. The solid particle according to claim 17, wherein the poorly soluble drug is in micronized form.

22. The solid particle according to claim 17, wherein the poorly soluble drug is in micronized form and has an average particle size of less than 50 μm.

23. The solid particle according to claim 17, wherein the poorly soluble drug is in micronized form and has an average particle size of less than 30 μm.

24. The solid particle according to claim 17, wherein the poorly soluble drug is in micronized form and has an average particle size of less than 20 μm.

25. The solid particle according claim 17, wherein the poorly soluble drug is in micronized form and has an average particle size of less than 10 μm.

26. The solid particle according to claim 17, wherein the solubilizer is selected from the group consisting of polyethylene oxide, hydroxyalkyl cellulose, hydroxypropylalkyl cellulose, polyvinyl alcohol, polyvinylpyrrolidone, copovidone, sodium carboxymethyl cellulose, carbopol, sodium alginate, xanthan gum, locust bean gum, cellulose gum, gellan gum, tragacanth gum, karaya gum, guar gum, acacia gum, poloxamer, cyclodextrin, dextrin derivatives, surfactants and mixtures thereof.

27. The solid particle according to claim 17, wherein the solubilizer is a surfactant selected from the group consisting of self-emulsifying glyceryl monooleate, docusate sodium, emulsifying wax BP, sodium lauryl sulfate, benzethonium chloride, cetrimide, cetylpyridinium chloride, lauric acid, myristyl alcohol, butylparaben, ethylparaben, methylparaben, propylparaben, sorbic acid, emulsifying wax, glyceryl monooleate, phospholipids, polyoxyethylene alkyl ethers (macrogol cetostearyl ether, macrogol lauryl ether, macrogol oleyl ether, macrogol stearyl ether), polyoxyethylene castor oil derivatives (macrogolglycerol ricinoleate, macrogolglycerol hydroxystearate), polyoxyethylene sorbitan fatty acid esters (polysorbate 20, 40, 60, and 80), polyoxytehylene stearates, polyoxylglycerides (caprylocaproyl polyoxylglycerides, lauroyl polyoxylglycerides, linoleoyl polyoxylglycerides, oleoyl polyoxylglycerides and stearoyl polyoxylglycerides), sorbitan esters (sorbitan laurate, sorbitan oleate, sorbitan palmitate, sorbitan sesquioleate, sorbitan stearate, sorbitan trioleate), triethyl citrate and mixtures thereof.

28. The solid particle according to claim 17, wherein the solubilizer is a surfactant selected from the group consisting of solid sodium lauryl sulfate, polyoxyethylene sorbitan fatty acid esters and polyoxylglycerides.

29. A process for producing solid particles according to claim 17, wherein said process comprises the following steps:
a. the solubilizer is dissolved or suspended in a polar or a non-polar solvent, protic or aprotic or mixtures thereof
b. the solution obtained in step a. is poured or sprayed on to the surface of the poorly soluble drug.

30. The process according to claim 29, wherein the solvent is water.

31. An oral pharmaceutical composition comprising solid particles according to claim 17 and at least one pharmaceutically acceptable excipient.

32. The oral pharmaceutical composition according to claim 31, wherein the solid particles comprise an anticoagulant agent.

33. The oral pharmaceutical composition according to claim 31, wherein the solid particles comprise rivaroxaban or apixaban.

34. The oral pharmaceutical composition according to claim 31 which is a tablet, a minitablet or an orodispesible tablet.

35. The oral pharmaceutical composition according to claim 34 prepared by direct compression.

36. A process for producing the oral pharmaceutical composition according to claim 31 comprising:
a. preparing the solid particles of a poorly soluble drug
b. mixing the particles of step a. with at least one pharmaceutical excipient

37. The process according to claim 36, further comprising pressing the mixture obtained in step (b) in to a tablet.

38. A method of prophilaxis and/or treatment of thromboembolic diseases, which method comprises administering to a patient in need of such treatment a therapeutically effective amount of an oral pharmaceutical composition according to claim 31.
